(19) United States
(12) Patent Application Publication
(10) Pub. No.: US 2015/0185226 A1
(43) Pub. Date: Jul. 2, 2015

(54) Method for Diagnosing Scleroderma

(71) Applicants: INSERM (Institut National de la Sante et la Recherche Medicale), Paris (FR); Assistance Publique-Hôpitaux de Marseille, Marseille (FR); Université d'Aix Marseille, Marseille Cedex 07 (FR)

(72) Inventors: Nathalie Lambert, Marseille Cedex 09 (FR); Isabelle Auger, Marseille Cedex 09 (FR); Jean Roudier, Marseille Cedex 09 (FR)

(21) Appl. No.: 14/416,334

(22) PCT Filed: Jul. 23, 2013
(86) PCT No.: PCT/EP2013/065490

§ 371 Date: Jan. 22, 2015

(30) Foreign Application Priority Data
Jul. 23, 2012 (EP) 12305893.5

(51) Int. Cl. G01N 33/68

(52) U.S. Cl. CPC G01N 33/6803; G01N 2800/24

(57) ABSTRACT

The present invention relates to an in vitro method for diagnosing scleroderma in a subject, said method comprising the step of detecting in a biological sample obtained from the subject one or more autoantibodies recognizing one or more protein biomarkers selected from the group of proteins consisting of THEX1, AIF1, FGF2, EphB2, CLK1 and ANKS6.

### FIELD OF THE INVENTION

The invention relates to an in vitro method for diagnosing scleroderma in a subject, said method comprising the step of detecting in a biological sample obtained from the subject one or more autoantibodies recognizing one or more protein biomarkers selected from the group of proteins consisting of THEX1, AIF1, FGF2, EphB2, CLK1 and ANKS6.

### BACKGROUND OF THE INVENTION

Systemic sclerosis (SSc) or scleroderma is a severe chronic autoimmune disease starting with vascular damages, triggering to collagen synthesis dysfunction and fibrotic phenotype in the skin and internal organs. SSc is a rather rare disease still difficult to diagnose because of its clinical heterogeneity among patients. Two main forms of systemic sclerosis are defined: limited cutaneous disease (lcSSc) including CREST syndrome (Calcinosis, Raynaud phenomenon, Esophageal dysmotility, Sclerodactily, and Telangiectasia) characterized by skin involvement contained below the elbows and knees and diffuse cutaneous disease (dcSSc) for which skin involvement extends over. There is another form of scleroderma called localized scleroderma by opposition to systemic. The localized form is often called morphea, not to be confounded with limited systemic sclerosis.

More than 80% of patients with SSc have anti-nuclear antibodies (ANA). Some specific autoantibodies correlate with clinical subtypes and are helpful for diagnosis and clinical classification. The presence of anti-centromere antibodies (ACA) is associated with manifestations of lcSSc and involvement of internal organs is at low frequency. The presence of anti-topoisomerase antibodies (ATA or Anti-Scl-70) is associated with the diffuse forms of cutaneous involvement, severity of interstitial lung disease and increased prevalence of right heart failure secondary to pulmonary disease. Therefore ACA and ATA are respectively a hallmark of lcSSc and dcSSc and represent respectively 65% and 40% of patients in each clinical subgroup [Allanore Y. et al., 2007].

Other biomarkers can be used for SSc classification, although less frequent and less frequently tested in routine. Anti-RNA polymerase III antibodies for example are good diagnostic markers for dcSSc in American cohorts (25% of SSc) but less in European cohorts (9% of SSc). Less often detected (1 to 5%) anti-nuclear (anti Th/To) antibodies are present in limited form of the disease. Other auto-antibodies: anti-ku, anti-Pm/Scl or anti-U1RNP are associated with overlapping syndromes, respectively polymyosites, scleromyosites and mixed connective tissue disease [Ho K T et al., 2003; Reveille J D, 2006; Arnett F C et al., 2010 and Walker U A et al., 2007].

Patent application WO2007013124 discloses a method for diagnosing autoimmune diseases and particularly systemic sclerosis in a subject comprising the step of detecting in a biological sample obtained from the subject an autoantibody recognizing the protein biomarkers Platelet Derived Growth Factor (PDGF).

Allograft Inflammatory Factor 1 (AIF1) has already been described as involved in SSc, but not in the context of being an autoantigen. The article from Alkassab et al. [Alkassab F. et al. 2007] discloses the association of Single Nucleotide Polymorphisms (SNPs) in the AIF gene and presence of anti-centromere antibodies in patients with systemic sclerosis.

About 30% of patients with SSc do not have one or the other classical autoantibodies found in SSc and are therefore difficult to be diagnosed or classified. Indeed, SSc is probably one of the disease for which specific biomarkers are the most useful for clinical evaluation and disease prognosis.

Deciphering patient's plasma to identify new biomarkers would allow finding better prognosis and diagnosis proteins but would also shed further light on a disease for which, up to now, there is no treatment to stop the course.

### SUMMARY OF THE INVENTION

In the current study, the inventors selected 6 protein candidates from ProtoArray® analysis with a very stringent approach and validated their specificity by ELISA on a larger group of patients and controls including other autoimmune diseases.

Thus, the invention relates to an in vitro method for diagnosing scleroderma in a subject, said method comprising the step of detecting in a biological sample obtained from the subject one or more autoantibodies recognizing one or more protein biomarkers selected from the group of proteins consisting of THEX1, AIF1, FGF2, EphB2, CLK1 and ANKS6.

### DETAILED DESCRIPTION OF THE INVENTION

In one aspect, the present invention provides an in vitro method for diagnosing scleroderma in a subject, said method comprising the step of detecting in a biological sample obtained from the subject one or more autoantibodies recognizing one or more protein biomarkers selected from the group of proteins consisting of THEX1, AIF1, FGF2, EphB2, CLK1 and ANKS6.

In one embodiment, the scleroderma is a systemic sclerosis (SSc).

In another embodiment, the systemic sclerosis is a limited cutaneous systemic sclerosis (IcSSc) or a diffuse cutaneous systemic sclerosis (dcSSc).

In one embodiment, the scleroderma is a localized scleroderma.

In particulars embodiments, a plurality of protein biomarkers (i.e., two or more than two protein biomarkers) is used in the method of diagnosis. In other words, the method of the invention may comprise steps of: contacting the biological sample with a plurality of protein biomarkers for a time and under conditions allowing protein biomarker-antibody complexes to form between two, three, four, five or six protein biomarker and autoantibodies present in the biological sample; and detecting any protein biomarker-antibody complex formed.

In particular embodiments, the method of diagnosis is performed using 6 different protein biomarkers including THEX1, AIF1, FGF2, EphB2, CLK1 and ANKS6, analogs thereof and fragments thereof as described herein.

In other particular embodiments, the method of diagnosis is performed using the following two protein biomarkers: THEX1 and AIF1.

In other particular embodiments, the method of diagnosis is performed using the following protein biomarker: THEX1.

In other particular embodiments, the method of diagnosis is performed using the following protein biomarker: AIF1.

In the methods of diagnosis provided herein, the step of detecting any protein biomarker-antibody complex formed between a protein biomarker and an autoantibody present in the biological sample may be performed by any suitable method. In certain embodiments, the detection is by immunoassay.

In particular embodiments, the protein biomarker or biomarkers used in the diagnosis method is/are immobilized on a solid carrier or support.

Typically, the methods of diagnosis may further comprise measuring, in a biological sample obtained from the subject, the concentration of at least one scleroderma biomarker also known for the diagnostic of scleroderma (see for example Steen V D et al., 1998; Okano Y et al., 1993; Okano Y et al., 1992; Okano Y et al., 1990 and Oddis C V et al., 1992), for detecting the presence of scleroderma-specific autoantibodies, such as anti-topoisomerase I, anti-centromere B, anti-ku, anti-Pm/Scl or anti-U1RNP.

In another aspect, the present invention provides kits for the in vitro diagnosis of scleroderma in a subject. These kits comprise: one, two, three, four, five or six protein biomarker of the invention and at least one reagent for detecting a protein biomarker-antibody complex formed between the protein biomarker and an autoantibody present in the biological sample to be tested. In the kits, the at least one protein biomarker may be immobilized on a solid carrier or support, or alternatively, reagents may be included in the kit that can be used to immobilize the protein biomarker on a solid carrier or support. The kits may further comprise instructions for carrying out a method of diagnosis according to the present invention. In certain embodiments, the kit comprises at least 6 protein biomarkers including THEX1, AIF1, FGF2, EphB2, CLK1 and ANKS6 proteins, or fragments thereof. In other embodiments, the kit comprises the following two biomarkers: THEX1 and AIF1.

In certain preferred embodiments, these kits further comprise at least one additional scleroderma biomarker also known for the diagnostic of scleroderma, for detecting the presence of scleroderma-specific autoantibodies as described in Steen V D et al., 1998; Okano Y et al., 1993; Okano Y et al., 1992; Okano Y et al., 1990 and Oddis C V et al., 1992. For example, the additional scleroderma biomarker also known for the diagnostic of scleroderma include, but are not limited to topoisomerase I, centromere B, ku, Pm/Scl or U1RNP.

In particular embodiments, these kits further comprise at least one additional scleroderma biomarker also known for the diagnostic of scleroderma as described below for detecting the presence of scleroderma-specific autoantibodies, such as anti-topoisomerase I, anti-centromere B, anti-ku, anti-Pm/Scl or anti-U1RNP.

In yet another aspect, the present invention provides arrays for the diagnosis of scleroderma in a subject. An array according to the invention comprises, attached to its surface, at least one protein biomarker of the invention. The array comprises, attached to its surface, at least 6 protein biomarkers including the THEX1, AIF1, FGF2, EphB2, CLK1 and ANKS6 proteins described herein. In certain embodiments, the array comprises, attached to its surface, the following two biomarkers: THEX1 and AIF1.

In certain preferred embodiments, an inventive array further comprises at least one additional scleroderma biomarker also known for the diagnostic of scleroderma, for detecting the presence of scleroderma-specific autoantibodies as described in Steen V D et al., 1998; Okano Y et al., 1993; Okano Y et al., 1992; Okano Y et al., 1990 and Oddis C V et al., 1992. For example, the additional scleroderma biomarker also known for the diagnostic of scleroderma include, but are not limited to topoisomerase I, centromere B, ku, Pm/Scl or anti-U1RNP.

In particular embodiments, the array further comprises, attached to its surface, at least one additional scleroderma biomarker also known for the diagnostic of scleroderma as described below for detecting the presence of scleroderma-specific autoantibodies, such as anti-topoisomerase I, anti-centromere B, anti-ku, anti-Pm/Scl or anti-U1RNP.

These and other objects, advantages and features of the present invention will become apparent to those of ordinary skill in the art having read the following detailed description of the preferred embodiments.

### DEFINITIONS

Throughout the specification, several terms are employed that are defined in the following paragraphs.

As used herein, the term “subject” refers to a human or another mammal (e.g., primate, dog, cat, goat, horse, pig, mouse, rat, rabbit, and the like), that can be afflicted with scleroderma. In a preferred embodiment of the present invention, the subject is a human being. In such embodiments, the subject is often referred to as an “individual”. The term “individual” does not denote a particular age, and thus encompasses children, teenagers, and adults.

The term “subject suspected of having scleroderma” refers to a subject that presents one or more symptoms indicative of scleroderma or scleroderma (e.g., skin symptoms like hardening and scarring, musculoskeletal, pulmonary, gastrointestinal, renal and other complications), or that is screened for scleroderma (e.g., during a physical examination). Alternatively or additionally, a subject suspected of having scleroderma may have one or more risk factors (e.g., age, sex, family history, smoking, etc). The term encompasses subjects that have not been tested for scleroderma as well as subjects that have received an initial diagnosis.

The term “biological sample” is used herein in its broadest sense. A biological sample is generally obtained from a subject. A sample may be of any biological tissue or fluid with which biomarkers of the present invention may be assayed. Frequently, a sample will be a “clinical sample”, i.e., a sample derived from a patient. Such samples include, but are not limited to, bodily fluids which may or may not contain cells, e.g., blood (e.g., whole blood, serum or plasma), synovial fluid, saliva, tissue or fine needle biopsy samples, and archival samples with known diagnosis, treatment and/or outcome history. Biological samples may also include sections of tissues such as frozen sections taken for histological purposes. The term “biological sample” also encompasses any material derived by processing a biological sample. Derived materials include, but are not limited to, cells (or their progeny) isolated from the sample, or proteins extracted from the sample. Processing of a biological sample may involve one or more of: filtration, distillation, extraction, concentration, inactivation of interfering components, addition of reagents, and the like.

In a preferred embodiment of the invention, the biological sample is a serologic sample and is or is derived from whole blood, serum or plasma obtained from a subject.

The terms “normal” and “healthy” are used herein interchangeably. They refer to a subject that has not presented any scleroderma symptoms or systemic sclerosis symptoms, and that has not been diagnosed with scleroderma or systemic sclerosis. Preferably, a normal subject is not on medication for scleroderma or systemic sclerosis and has not been diagnosed with any other autoimmune disease and had no family history of autoimmunity. In certain embodiments, normal subjects may have similar sex, age, and/or body mass index as compared with the subject from which the biological sample to be tested was obtained. The term “normal” is also used herein to qualify a sample obtained from a healthy subject.

In the context of the present invention, the term “control”, when used to characterize a subject, refers to a subject that is healthy or to a patient who has been diagnosed with a specific autoimmune disease other than scleroderma. The term “control sample” refers to one, or more than one, sample that has been obtained from a healthy subject or from a patient diagnosed with an autoimmune disease other than scleroderma.

The term “autoantibody”, as used herein, has meaning accepted in the art, and refers to an antibody that is produced by the immune system of a subject and that is directed against subject's own proteins. Autoantibodies may attack the body's own cells, tissues, and/or organs, causing inflammation and damage.

As used herein, the term “autoantigen” refers to an endogenous antigen, or an active fragment thereof, that stimulates the production of autoantibodies in a body of a subject, as in autoimmune reactions. The term also encompasses any substances that can form an antigen-antibody complex with autoantibodies present in a subject or in a biological sample obtained from a subject.

The terms “biomarker”, “protein biomarker” and “marker” are used herein interchangeably. They refer to a substance that is a distinctive indicator of a biological process, biological event, and/or pathologic condition. In the context of the present invention, the term “biomarker of scleroderma” or “scleroderma biomarker” encompasses THEX1, AIF1, FGF2, EphB2, CLK1 and ANKS6 proteins provided herein which are specifically recognized by anti-THEX1 autoantibodies, anti-AIF1 autoantibodies, anti-FGF2 autoantibodies, anti-EphB2 autoantibodies, anti-CLK1 autoantibodies, and anti-ANKS6 autoantibodies present in a biological sample (e.g., blood sample) of a scleroderma patient. In certain preferred embodiments, the biomarkers of the invention are proteins fragment of less than 20 amino acids. In more preferred embodiments, the biomarkers of the invention are proteins fragment of between 5 and 20 amino acids (i.e. 10 or 15 amino acids).

As used herein, the term “indicative of scleroderma”, when applied to a process or event, refers to a process or event which is a diagnostic of scleroderma, such that the process or event is found significantly more often in subjects with scleroderma than in healthy subjects and/or in subjects suffering from a disease other than scleroderma.

The terms “protein”, “polypeptide”, and “peptide” are used herein interchangeably, and refer to amino acid sequences of a variety of lengths, either in their neutral (uncharged) forms or as salts, and either unmodified or modified by glycosylation, side chain oxidation, or phosphorylation, or citrullination. In certain embodiments, the amino acid sequence is a full-length native protein. In other embodiments, the amino acid sequence is a smaller fragment of the full-length protein. In still other embodiments, the amino acid sequence is modified by additional substituents attached to the amino acid side chains, such as glycosyl units, lipids, or inorganic ions such as phosphates, as well as modifications relating to chemical conversion of the chains such as oxidation of sulfhydryl groups. Thus, the term “protein” (or its equivalent terms) is intended to include the amino acid sequence of the full-length native protein, or a fragment thereof, subject to those modifications that do not significantly change its specific properties. In particular, the term “protein” encompasses protein isoforms, i.e., variants that are encoded by the same gene, but that differ in their pI or MW, or both. Such isoforms can differ in their amino acid sequence (e.g., as a result of alternative splicing or limited proteolysis), or in the alternative, may arise from differential post-translational modification (e.g., glycosylation, acylation, phosphorylation).

The term “analog”, when used herein in reference to a protein or polypeptide, refers to a peptide that possesses a similar or identical function as the protein or polypeptide but need not necessarily comprise an amino acid sequence that is similar or identical to the amino acid sequence of the protein or polypeptide or a structure that is similar or identical to that of the protein or polypeptide. Preferably, in the context of the present invention, an analog has an amino acid sequence that is at least 80%, more preferably, at least about: 80%, 85%, 90%, 95%, 96%, 97%, 98% or 99%, identical to the amino acid sequence of the protein or polypeptide. In certain preferred embodiments, an analog of a peptide biomarker of the invention has an amino acid sequence that is at least 80% identical or at least 85% identical to the amino acid sequence of the peptide biomarker.

The term “homologous” (or “homology”), as used herein, is synonymous with the term “identity” and refers to the sequence similarity between two polypeptide molecules or between two nucleic acid molecule. When a position in both compared sequences is occupied by the same base or same amino acid residue, then the respective molecules are homologous at that position. The percentage of homology between two sequences corresponds to the number of matching or homologous positions shared by the two sequences divided by the number of positions compared and multiplied by 100. Generally, a comparison is made when two sequences are aligned to give maximum homology. Homologous amino acid sequences share identical or similar amino acid sequences. Similar residues are conservative substitutions for, or “allowed point mutations” of, corresponding amino acid residues in a reference sequence. “Conservative substitutions” of a residue in a reference sequence are substitutions that are physically or functionally similar to the corresponding reference residue, e.g., that have a similar size, shape, electric charge, chemical properties, including the ability to form covalent or hydrogen bonds, or the like. Particularly preferred conservative substitutions are those fulfilling the criteria defined for an “accepted point mutation” by Dayhoff et al. (“Atlas of Protein Sequence and Structure”, 1978, Nat. Biomed. Res. Foundation, Washington, D.C., Suppl. 3, 22: 354-352).

The terms “labeled”, “labeled with a detectable agent” and “labeled with a detectable moiety” are used herein interchangeably. These terms are used to specify that an entity (e.g., a THEX1, AIF1, FGF2, EphB2, CLK1 and ANKS6 proteins) can be visualized, for example, following binding to another entity (e.g., an anti-THEX1 autoantibodies, anti-AIF1 autoantibodies, anti-FGF2 autoantibodies, anti-EphB2 autoantibodies, anti-CLK1 autoantibodies and anti-ANKS6 autoantibodies). Preferably, a detectable agent or moiety is selected such that it generates a signal which can be measured and whose intensity is related to the amount of bound entity. In array-based methods, a detectable agent or moiety is also preferably selected such that it generates a localized signal, thereby allowing spatial resolution of the signal from each spot on the array. Methods for labeling proteins and polypeptides are well-known in the art. Labeled polypeptides can be prepared by incorporation of or conjugation to a label, that is directly or indirectly detectable by spectroscopic, photochemical, biochemical, immunochemical, electrical, optical, or chemical means, or any other suitable means. Suitable detectable agents include, but are not limited to, various ligands, radionuclides, fluorescent dyes, chemiluminescent agents, microparticles, enzymes, colorimetric labels, magnetic labels, and haptens.

The terms “protein array” and “protein chip” are used herein interchangeably. They refer to a substrate surface on which different proteins or polypeptides are immobilized, in an ordered manner, at discrete spots on the substrate. Protein arrays may be used to identify protein/protein interactions (e.g., antigen/antibody interactions), to identify the substrates of enzymes, or to identify the targets of biologically active small molecules. The term “microarray” specifically refers to an array that is miniaturized so as to require microscopic examination for visual evaluation.

The term “THEX1”, refers to the protein named “three prime histone mRNA exonuclease 1”. The sequence of said protein may be found under the NCBI Reference: NM_{—}153332.2. Protein THEX1 identified as described herein has the following amino acid sequence:

SEQ ID No 1:
MEDPQSKEPAGEAVALALLESPRPEGGEEPPRPSPEETQQCKFDGQETK
GSKFITSSASDFSDPVYKEIAITNGCINRMSKEELRAKLSEFKLETRGV
KDVLKKRLKNYYKKQKLMLKESNFADSYYDYICIIDFEATCEEGNPPEF
VHEIIEFPVVLLNTHTLEIEDTFQQYVRPEINTQLSDFCISLTGITQDQ
VDRADTFPQVLKKVIDWMKLKELGTKYKYSLLTDGSWDMSKFLNIQCQL
SRLKYPPFAKKWINIRKSYGNFYKVPRSQTKLTIMLEKLGMDYDGRPHC
GLDDSKNIARIAVRMLQDGCELRINEKMHAGQLMSVSSSLPIEGTPPPQ
MPHFRK.

To be understood broadly, the term “THEX1” refers to the protein and also to analogs and fragments of the protein. The term “THEX1 fragment”, refers to a peptide comprising an amino acid sequence of at least 5 amino acid residues (preferably, of at least: 10, 15, 20, 25, 30, 40, 50, 60, or 70 amino acid residues) of the amino acid sequence of a THEX1 protein. In preferred embodiments of the present invention, a fragment of THEX1 biomarker of the invention comprises an amino acid sequence of at least 5 consecutive amino acid residues of the amino acid sequence of the peptide biomarker and is not the whole protein.

The term “AIF1”, refers to the protein named “Allograft inflammatory factor 1”. The sequence of said protein may be found under the NCBI Reference: NM_{—}032955.1. Protein AIF1 identified as described herein has the following amino acid sequence:

SEQ ID No 2:
MEFDLNGNGDIDIMSLKRMLEKLGVPKTHLELKKLIGEVSSGSGETFSY
PDFLRMMLGKRSAILKMILMYEEKAREKEKPTGPPAKKAISELP.

To be understood broadly, the term “AIF1” refers to the protein and also to analogs and fragments of the protein. The term “AIF1 fragment”, refers to a peptide comprising an amino acid sequence of at least 5 amino acid residues (preferably, of at least: 10, 15, 20, 25, 30, 40, 50, 60, 70, 80, 90, 100, 125, 150, 175, 200, or 250 amino acid residues) of the amino acid sequence of a AIF1 protein. In preferred embodiments of the present invention, a fragment of AIF1 biomarker of the invention comprises an amino acid sequence of at least 5 consecutive amino acid residues of the amino acid sequence of the peptide biomarker and is not the whole protein.

The term “FGF2”, refers to the protein named “fibroblast growth factor 2” also known as “Basic fibroblast growth factor (bFGF)”. The sequence of said protein may be found with the NCBI Reference: NM_{—}002006.3. Protein FGF2 identified as described herein has the following amino acid sequence:

SEQ ID No 3:
MAAGSITTLPALPEDGGSGAFPPGHFKDPKRLYCKNGGFFLRIHPDGRV
DGVREKSDPHIKLQLQAEERGVVSIKGVCANRYLAMKEDGRLLASKCVT
DECFFFERLESNNYNTYRSRKYTSWYVALKRTGQYKLGSKTGPGQKAIL
FLPMSAKS.

To be understood broadly, the term “FGF2” refers to the protein and also to analogs and fragments of the protein. The term “FGF2 fragment”, refers to a peptide comprising an amino acid sequence of at least 5 amino acid residues (preferably, of at least: 10, 15, 20, 25, 30, 40, 50, 60, 70, 80, 90, 100, 125, 150, 175, or 200 amino acid residues) of the amino acid sequence of a FGF2 protein. In preferred embodiments of the present invention, a fragment of FGF2 biomarker of the invention comprises an amino acid sequence of at least 5 consecutive amino acid residues of the amino acid sequence of the peptide biomarker and is not the whole protein.

The term “EphB2”, refers to the protein named “Ephrin type-B receptor 2”. The sequence of said protein may be found under the NCBI Reference: NM_{—}004442.3. Protein EphB2 identified as described herein has the following amino acid sequence:

SEQ ID No 4:
HNQTSLYKKAGSEIDISCVKIEQVIGAGEFGEVCSGHLKLPGKREIFVA
IKTLKSGYTEKQRRDFLSEASIMGQFDHPNVIHLEGVVTKSTPVMIITE
FMENGSLDSFLRQNDGQFTVIQLVGMLRGIAAGMKYLADMNYVHRDLAA
RNILVNSNLVCKVSDFGLSRFLEDDTSDPTYTSALGGKIPIRWTAPEAI
QYRKFTSASDVWSYGIVMWEVMSYGERPYWDMTNQDVINAIEQDYRLPP
PMDCPSALHQLMLDCWQKDRNHRPKFGQIVNTLDKMIRNPN.

To be understood broadly, the term “EphB2” refers to the protein and also to analogs and fragments of the protein. The term “EphB2 fragment”, refers to a peptide comprising an amino acid sequence of at least 5 amino acid residues (preferably, of at least about: 10, 15, 20, 25, 30, 40, 50, 60, 70, 80, 90, 100, 125, 150, 175, 200, or 250 amino acid residues) of the amino acid sequence of a EphB2 protein. In preferred embodiments of the present invention, a fragment of EphB2 biomarker of the invention comprises an amino acid sequence of at least 5 consecutive amino acid residues of the amino acid sequence of the peptide biomarker and is not the whole protein.

The term “CLK1”, refers to the protein named “dual specificity protein kinase CLK1 isoform 1”. The sequence of said protein may be found under the NCBI Reference: NM_{—}004071.1. Protein CLK1 identified as described herein has the following amino acid sequence:

SEQ ID No 5:
MSPILGYWKIKGLVQPTRLLLEYLEEKYEEHLYERDEGDKWRNKKFELG
LEFPNLPYYIDGDVKLTQSMAIIRYIADKHNMLGGCPKERAEISMLEGA
VLDIRYGVSRIAYSKDFETLKVDFLSKLPEMLKMFEDRLCHKTYLNGDH
VTHPDFMLYDALDVVLYMDPMCLDAFPKLVCFKKRIEAIPQIDKYLKSS
KYIAWPLQGWQATFGGGDHPPKSDLVPRPWSNQTSLYKKAGFEGDRTMH
GKSHRRKRTRSVEDDEEGHLICQSGDVLSARYEIVDTLGEGAFGKVVEC
IDHKAGGRHVAVKIVKNVDRYCEAARSEIQVLEHLNTTDPNSTFRCVQM
LEWFEHHGHICIVFELLGLSTYDFIKENGFLPFRLDHIRKMAYQICKSV
NFLHSNKLTHTDLKPENILFVQSDYTEAYNPKIKRDERTLINPDIKVVD
FGSATYDDEHHSTLVSTRHYRAPEVILALGWSQPCDVWSIGCILIEYYL
GFTVFPTHDSKEHLAMMERILGPLPKHMIQKTRKRKYFHHDRLDWDEHS
SAGRYVSRRCKPLKEFMLSQDVEHERLFDLIQKMLEYDPAKRITLREAL
KHPFFDLLKKSI.

To be understood broadly, the term “CLK1” refers to the protein and also to analogs and fragments of the protein. The term “CLK1 fragment”, refers to a peptide comprising an amino acid sequence of at least 5 amino acid residues (preferably, of at least: 10, 15, 20, 25, 30, 40, 50, 60, 70, 80, 90, 100, 125, 150, 175, or 200 amino acid residues) of the amino acid sequence of a CLK1 protein. In preferred embodiments of the present invention, a fragment of CLK1 biomarker of the invention comprises an amino acid sequence of at least 5 consecutive amino acid residues of the amino acid sequence of the peptide biomarker and is not the whole protein.

The term “ANKS6”, refers to the protein named “ankyrin repeat and sterile alpha motif domain containing 6”. The sequence of said protein may be found under the NCBI Reference: BC012981.2. Protein P6 identified as described herein has the following amino acid sequence:

SEQ ID No 6:
MPIRDDISWPPSAVCSVSLILHLPGRFGHVSVAHLLLDHGADVNAQNRL
GASVLTVASRGGHLGVVKLLLEAGAFVDHHHPSGEQLGLGGSRDEPLDI
TALMAAIQHGHEAVVRLLMEWGADPNHAARTVGWSPLMLAALTGRLGVA
QQLVEKGANPDHLSVLEKTAFEVALDCKHRDLVDYLDPLTTVRPKTGQA
ACPPWLHRGPQIVFMWLKLRIALLEGHAELRVQPCRPLRLRKWCA.

To be understood broadly, the term “ANKS6” refers to the protein and also to analogs and fragments of the protein. The term “ANKS6 fragment”, refers to a peptide comprising an amino acid sequence of at least 5 amino acid residues (preferably, of at least: 10, 15, 20, 25, 30, 40, 50, 60, 70, 80, 90, 100, 125, 150, 175, or 200 amino acid residues) of the amino acid sequence of a ANKS6 protein. In preferred embodiments of the present invention, a fragment of ANKS6 biomarker of the invention comprises an amino acid sequence of at least 5 consecutive amino acid residues of the amino acid sequence of the peptide biomarker and is not the whole protein.

### DETAILED DESCRIPTION OF CERTAIN PREFERRED EMBODIMENTS

As mentioned above, the present invention provides biomarkers that can be used for detecting the presence of scleroderma-specific autoantibodies in biological samples obtained from patients. These biomarkers are THEX1, AIF1, FGF2, EphB2, CLK1 and ANKS6 proteins which respectively and specifically react with anti-THEX1 autoantibodies, anti-AIF1 autoantibodies, anti-FGF2 autoantibodies, anti-EphB2 autoantibodies, anti-CLK1 autoantibodies, and anti-ANKS6 autoantibodies present in the serum or plasma of scleroderma patients.

Other scleroderma biomarker also known for the diagnostic of scleroderma as described in Steen V D et al., 1998; Okano Y et al., 1993; Okano Y et al., 1992; Okano Y et al., 1990 and Oddis C V et al., 1992 may be used in the diagnostic method of the present invention are. For example, the additional scleroderma biomarker also known for the diagnostic of scleroderma may be selected in the group consisting of topoisomerase I, centromere B, ku, Pm/Scl or U1RNP.

The invention also provides methods for using these biomarkers in the diagnosis of systemic sclerosis.

I—Protein Biomarkers

Preparation of the Peptide Biomarkers

The polypeptide/protein biomarkers of the present invention may be prepared by any suitable method, including recombinant methods. Such methods, as described, for example, in “The Proteins” (Vol. II, 3rd Ed., H. Neurath et al. (Eds.), 1976, Academic Press: New York, N.Y., pp. 105-237) may also be used to synthesize the biomarkers of the invention.

In certain embodiments, a polypeptide/protein biomarker of the invention is provided which is immobilized onto a solid carrier or support (e.g., a bead or array). Methods for immobilizing polypeptide molecules onto a solid surface are known in the art. A polypeptide/protein may be immobilized by being either covalently or passively bound to the surface of a solid carrier or support. Examples of suitable carrier or support materials include, but are not limited to, agarose, cellulose, nitrocellulose, dextran, Sephadex, Sepharose, carboxymethyl cellulose, polyacrylamides, polystyrene, polyvinyl chloride, polypropylene, filter paper, magnetite, ion-exchange resin, glass, polyamine-methyl-vinyl-ether-maleic acid copolymer, amino acid copolymer, ethylene-maleic acid copolymer, nylon, silk, and the like. Immobilization of a polypeptide/protein biomarker on the surface of a solid carrier or support may involve crosslinking, covalent binding or physical adsorption, using methods well known in the art. The solid carrier or support may be in the form of a bead, a particle, a microplate well, an array, a cuvette, a tube, a membrane, or any other shape suitable for conducting a diagnostic method according to the invention (e.g., using an immunoassay).

In particular, the invention provides an array or protein array for the diagnosis of scleroderma, comprising, immobilized to its surface, at least one peptide biomarker of the invention. Preferably, the array comprises more than one polypeptide/protein biomarker of the invention.

The array may further comprises at least one additional scleroderma biomarker also known for the diagnostic of scleroderma, for detecting the presence of scleroderma-specific autoantibodies as described in Steen V D et al., 1998; Okano Y et al., 1993; Okano Y et al., 1992; Okano Y et al., 1990 and Oddis C V et al., 1992. For example, the additional scleroderma biomarker also known for the diagnostic of scleroderma may be selected in the group consisting of topoisomerase I, centromere B, ku, Pm/Scl or anti-U1RNP.

The present invention also provides a protein bead suspension array for the diagnosis of scleroderma This bead suspension array comprises a suspension of one or more identifiable distinct particles or beads, wherein each bead contains coding features relating to its size, color or fluorescence signature and wherein each bead is coated with a polypeptide/protein biomarker of the present invention. Examples of bead suspension arrays include thexMAP® bead suspension array (Luminex Corporation).

II—Methods of Diagnosis

The present invention provides methods for the diagnosis of scleroderma in a subject. Such methods comprise contacting a biological sample obtained from the subject to be tested with one, two, three, four, five or six biomarkers for a time and under conditions allowing a biomarker-antibody complex to form; and detecting the biomarker-antibody complexes formed.

The biomarker may be selected from the group consisting of THEX1, AIF1, FGF2, EphB2, CLK1 and ANKS6.

In this method, the detection of a biomarker-antibody complex is indicative of scleroderma in the subject.

In one embodiment, the detection of a biomarker-antibody complex is indicative of systemic sclerosis in the subject.

The present invention provides methods for the diagnosis of scleroderma in a subject. In a particular embodiment, the scleroderma is a localized scleroderma.

In other embodiments, more than two biomarkers are used in combination, for example 3, 4, 5 or 6 biomarkers. In preferred embodiments, the combinations of biomarkers contain at least THEX1 and AIF1.

In certain preferred embodiments, 6 biomarkers are used, i.e., THEX1, AIF1, FGF2, EphB2, CLK1 and ANKS6.

In other embodiments, 2 biomarkers are used, i.e., THEX1 and AIF1.

In another embodiment, the method according to the invention the biological sample obtained from the subject is in contact with at least the 6 biomarkers and one or more protein biomarkers selected from the group of proteins consisting of the topoisomerase I, the centromere B, or the nuclear protein.

In a particular embodiment, the subject of the invention has no autoantibodies recognizing one or more protein biomarkers selected from the group of proteins consisting of topoisomerase I, the centromere B, or the nuclear protein.

In one embodiment, the invention relates to a method for diagnosing scleroderma in a subject, said method comprising the step of detecting at least one autoantibody selected from the group consisting of anti-THEX1 autoantibodies, anti-AIF1 autoantibodies, anti-FGF2 autoantibodies, anti-EphB2 autoantibodies, anti-CLK1 autoantibodies, and anti-ANKS6 autoantibodies.

In another embodiment, said method comprising the step of detecting at least one autoantibody selected from the group consisting of anti-THEX1 autoantibodies and anti-AIF1 autoantibodies.

Biological Samples

The method of diagnosis of the present invention may be applied to any type of biological sample allowing one or more biomarkers to be assayed. Examples of suitable biological samples include, but are not limited to, whole blood, serum, plasma, saliva, and synovial fluid. Biological samples used in the practice of the invention may be fresh or frozen samples collected from a subject, or archival samples with known diagnosis, treatment and/or outcome history. Biological samples may be collected by any non-invasive means, such as, for example, by drawing blood from a subject, or using fine needle aspiration or needle biopsy. In certain embodiments, the biological sample is a serologic sample and is selected from the group consisting of whole blood, serum, plasma.

In preferred embodiments, the inventive methods are performed on the biological sample itself without, or with limited, processing of the sample.

However, alternatively, the inventive methods may be performed on a protein extract prepared from the biological sample. In this case, the protein extract preferably contains the total protein content. Methods of protein extraction are well known in the art (see, for example “Protein Methods”, D. M. Bollag et al., 2nd Ed., 1996, Wiley-Liss; “Protein Purification Methods: A Practical Approach”, E. L. Harris and S. Angal (Eds.), 1989; “Protein Purification Techniques: A Practical Approach”, S. Roe, 2nd Ed., 2001, Oxford University Press; “Principles and Reactions of Protein Extraction, Purification, and Characterization”, H. Ahmed, 2005, CRC Press: Boca Raton, Fla.). Various kits can be used to extract proteins from bodily fluids and tissues. Such kits are commercially available from, for example, BioRad Laboratories (Hercules, Calif.), BD Biosciences Clontech (Mountain View, Calif.), Chemicon International, Inc. (Temecula, Calif.), Calbiochem (San Diego, Calif.), Pierce Biotechnology (Rockford, Ill.), and Invitrogen Corp. (Carlsbad, Calif.). User Guides that describe in great detail the protocol to be followed are usually included in all these kits. Sensitivity, processing time and costs may be different from one kit to another. One of ordinary skill in the art can easily select the kit(s) most appropriate for a particular situation.

Detection of Biomarker-Antibody Complexes

The diagnostic methods of the present invention involve detection of a biomarker-antigen complex formed between the protein biomarker and an autoantibody present in the biological sample tested. In the practice of the invention, detection of such a complex may be performed by any suitable method (see, for example, E. Harlow and A. Lane, “Antibodies: A Laboratories Manual”, 1988, Cold Spring Harbor Laboratory: Cold Spring Harbor, N.Y.).

For example, detection of a biomarker-antibody complex may be performed using an immunoassay. A wide range of immunoassay techniques is available, including radioimmunoassay, enzyme immunoassays (EIA), enzyme-linked immunosorbent assays (ELISA), and immunofluorescence immunoprecipitation. Immunoassays are well known in the art. Methods for carrying out such assays as well as practical applications and procedures are summarized in textbooks. Examples of such textbooks include P. Tijssen, In: Practice and theory of enzyme immunoassays, eds. R. H. Burdon and v. P. H. Knippenberg, Elsevier, Amsterdam (1990), pp. 221-278 and various volumes of Methods in Enzymology, Eds. S. P. Colowick et al., Academic Press, dealing with immunological detection methods, especially volumes 70, 73, 74, 84, 92 and 121. Immunoassays may be competitive or non-competitive.

For example, any of a number of variations of the sandwich assay technique may be used to perform an immunoassay. Briefly, in a typical sandwich assay applied to the detection of, for example, anti-THEX1 autoantibodies according to the present invention, an unlabeled THEX1-protein/polypeptide biomarker is immobilized on a solid surface (as described above) and the biological sample to be tested is brought into contact with the bound biomarker for a time and under conditions allowing formation of a biomarker-antibody complex. Following incubation, an antibody that is labelled with a detectable moiety and that specifically recognizes antibodies from the species tested (e.g., an anti-human IgG for human subjects) is added and incubated under conditions allowing the formation of a ternary complex between any biomarker-bound autoantibody and the labelled antibody. Any unbound material is washed away, and the presence of any anti-THEX1 autoantibody in the sample is determined by observation/detection of the signal directly or indirectly produced by the detectable moiety. Variations on this assay include an assay, in which both the biological sample and the labeled antibody are added simultaneously to the immobilized THEX1-protein/polypeptide biomarker.

The second antibody (i.e., the antibody added in a sandwich assay as described above) may be labeled with any detectable moiety, i.e., any entity which, by its chemical nature, provides an analytically identifiable signal allowing detection of the ternary complex, and consequently detection of the biomarker-antibody complex.

Detection may be either qualitative or quantitative. Methods for labelling biological molecules such as antibodies are well-known in the art (see, for example, “Affinity Techniques. Enzyme Purification: Part B”, Methods in Enzymol., 1974, Vol. 34, W. B. Jakoby and M. Wilneck (Eds.), Academic Press: New York, N.Y.; and M. Wilchek and E. A. Bayer, Anal. Biochem., 1988, 171: 1-32).

The most commonly used detectable moieties in immunoassays are enzymes and fluorophores. In the case of an enzyme immunoassay (EIA or ELISA), an enzyme such as horseradish perodixase, glucose oxidase, beta-galactosidase, alkaline phosphatase, and the like, is conjugated to the second antibody, generally by means of glutaraldehyde or periodate. The substrates to be used with the specific enzymes are generally chosen for the production of a detectable color change, upon hydrolysis of the corresponding enzyme. In the case of immunofluorescence, the second antibody is chemically coupled to a fluorescent moiety without alteration of its binding capacity. After binding of the fluorescently labeled antibody to the biomarker-antibody complex and removal of any unbound material, the fluorescent signal generated by the fluorescent moiety is detected, and optionally quantified. Alternatively, the second antibody may be labeled with a radioisotope, a chemiluminescent moiety, or a bioluminescent moiety.

Scleroderma Diagnosis

In the method of the present invention, detection of a biomarker-antibody complex is indicative of the presence of THEX1 autoantibodies, AIF1 autoantibodies, FGF2 autoantibodies, EphB2 autoantibodies, CLK1 autoantibodies or ANKS6 autoantibodies in the biological sample tested and is therefore indicative of scleroderma in the subject from which the biological sample is obtained. Thus, the methods of the present invention may be used for the diagnosis of scleroderma in patients. In particular, the method of the invention may be used for testing subjects suspected of having scleroderma.

In another embodiment, the method of the invention may be used for the diagnosis of systemic sclerosis in patients.

It will be appreciated by one skilled in the art that diagnosis of scleroderma may be performed solely on the basis of the results obtained by a method provided herein. Alternatively, a physician may also consider other clinical or pathological parameters used in existing methods to diagnose scleroderma. Thus, results obtained using methods of the present invention may be compared to and/or combined with results from other tests, assays or procedures performed for the diagnosis of scleroderma. Such comparison and/or combination may help provide a more refine diagnosis.

For example, scleroderma diagnosis methods of the present invention may be used in combination with scleroderma criteria (see for review Hachulla E et al. 2011).

Alternatively or additionally, results from scleroderma diagnosis methods of the present invention may be used in combination with results from one or more assays that employ other scleroderma biomarkers. Thus, in certain embodiments, diagnosis of scleroderma may be based on results from a method of the invention and on results from one or more additional assays that use a different scleroderma biomarker. For example, a panel of scleroderma biomarkers may be tested either individually or simultaneously, e.g., using a chip or a bead-based array technology.

Examples of suitable scleroderma biomarkers include scleroderma biomarker also known for the diagnostic of scleroderma as described in Steen V D et al., 1998; Okano Y et al., 1993; Okano Y et al., 1992; Okano Y et al., 1990 and Oddis C V et al., 1992. For example, suitable scleroderma biomarker also known for the diagnostic of scleroderma include, but are not limited to topoisomerase I, centromere B, ku, Pm/Scl or U1RNP.

In another embodiment, the method according to the invention the biological sample obtained from the subject is in contact with at least the 6 biomarkers of the invention and one or more protein biomarkers selected from the group of proteins consisting of a scleroderma biomarker also known for the diagnostic of scleroderma as described in Steen V D et al., 1998; Okano Y et al., 1993; Okano Y et al., 1992; Okano Y et al., 1990 and Oddis C V et al., 1992. For example, suitable scleroderma biomarker also known for the diagnostic of scleroderma include, but are not limited to topoisomerase I, centromere B, ku, Pm/Scl or U1RNP.

III—Kits

In another aspect, the present invention provides kits comprising materials useful for carrying out a diagnostic method according to the present invention. The diagnosis procedures provided herein may be performed by diagnostic laboratories, experimental laboratories, or practitioners. The invention provides kits that can be used in these different settings.

Materials and reagents for detecting anti-THEX1 autoantibodies, anti-AIF1 autoantibodies, anti-FGF2 autoantibodies, anti-EphB2 autoantibodies, anti-CLK1 autoantibodies, and anti-ANKS6 autoantibodies or any combination thereof in a biological sample and/or for diagnosing scleroderma, in a subject according to the present invention may be assembled together in a kit. Each kit of the invention comprises at least one protein/polypeptide biomarker of the invention preferably in an amount that is suitable for detection of autoantibodies in a biological sample.

Thus, in certain embodiments, a kit of the invention comprises one, two, three, four, five or six biomarkers that are selected from the group consisting of THEX1, AIF1, FGF2, EphB2, CLK1 and ANKS6.

In yet another embodiment, a kit of the invention comprises at least two, three, four, five, six biomarkers selected from the group consisting of: THEX1, AIF1, FGF2, EphB2, CLK1 and ANKS6.

In a preferred embodiment, the kit of the invention contains at least THEX1 or AIF1.

In another preferred embodiment, the kit comprises at least six biomarkers: THEX1, AIF1, FGF2, EphB2, CLK1 and ANKS6.

In yet another preferred embodiment, the kit comprises the two following biomarkers: THEX1 and AIF1.

The peptide biomarker(s) included in a kit may or may not be immobilized on the substrate surface (e.g., beads, array, and the like). Thus, in preferred embodiments, a kit of the invention includes an array for diagnosing scleroderma as provided herein. Alternatively, a substrate surface may be included in a kit of the invention for immobilization of the peptide bio markers.

A kit of the invention generally also comprises at least one reagent for the detection of a biomarker-antibody complex formed between the peptide biomarker included in the kit and an autoantibody present in a biological sample. Such a reagent may be, for example, a labelled antibody that specifically recognizes antibodies from the species tested (e.g., an anti-human IgG for human subjects), as described above.

Depending on the procedure, the kit may further comprise one or more of the following: extraction buffer and/or reagents, blocking buffer and/or reagents, immuno-detection buffer and/or reagents, labelling buffer and/or reagents, and detection means. Protocols for using these buffers and reagents for performing different steps of the procedure may be included in the kit.

The different reagents included in the kit of the invention may be supplied in a solid (e.g., lyophilized) or liquid form. The kits of the present invention may optionally comprise different containers (e.g., vial, ampoule, test tube, flask or bottle) for each individual buffer and/or reagent. Each component will generally be suitable as aliquoted in its respective container or provided in a concentrated form. Other containers suitable for conducting certain steps of the disclosed methods may also be provided. The individual containers of the kit are preferably maintained in close confinement for commercial sale.

In certain embodiments, a kit comprises instructions for using its components for the diagnosis of scleroderma, in a subject according to a method of the invention. Instructions for using the kit according to methods of the invention may comprise instructions for processing the biological sample obtained from the subject and/or for performing the test, and/or instructions for interpreting the results. A kit may also contain a notice in the form prescribed by a governmental agency regulating the manufacture, use or sale of pharmaceuticals or biological products.

The invention will be further illustrated by the following figures and examples. However, these examples and figures should not be interpreted in any way as limiting the scope of the present invention.

### FIGURES

FIG. 1: ELISA's titration of AIF1.

*p=0.04 when all three groups are compared (ANOVA test). The horizontal black line represents a ΔOD threshold at 0.055 to allow stringent discrimination for SSc versus other autoimmune diseases and healthy status.

FIG. 2: ELISA's titration of THEX1.

*p=0.001 when all three groups are compared (ANOVA test). The horizontal black line represents a ΔOD threshold at 0.08 to allow stringent discrimination for SSc versus other autoimmune diseases and healthy status.

TABLE 1
Number of recognized proteins by ProtoArray method.

| | Number of | |
|---|---|---|
| | recognized proteins | *P values |
| All patients with SSc (N = 20) | 4221 | ^{a}p = 0.0014 |
| | | ^{b}p = 0.058 |
| | | ^{c}p = 0.0009 |
| Abneg (N = 8) | 4438 | |
| ATA (N = 6) | 3562 | |
| ACA (N = 6) | 4590 | |
| All controls (N = 18) | 2467 | |
| Other AID (N = 10) | 3080 | ^{c}p = 0.029 |
| Healthy controls (N = 8) | 1685 | |

*P values from Mann Whitney test, ^{a}compared with all controls, ^{b}compared with other AID,^{c}compared with healthy controls.

TABLE 2
Protoarray analysis of the six candidate proteins recognized by
at least half of patients with SSc and none of the controls.

| ² | FGF2 | AIF1 | EphB2 | CLK1 | THEX1 | ANKS6 |
|---|---|---|---|---|---|---|
| Total SSc | 11 (55.0%) | 10 (50.0%) | 10 (50.0%) | 10 (50.0%) | 10 (50.0%) | 10 (50.0%) |
| (N = 20) | | | | | | |
| Ab^{neg}SSc | 2 (25.0%) | 4 (50.0%) | 1 (12.5%) | 2 (25.0%) | 5 (62.5%) | 4 (50.0%) |
| (N = 8) | | | | | | |
| ATA SSc | 6 (85.7%) | 4 (57.1%) | 6 (85.7%) | 6 (85.7%) | 3 (42.9%) | 3 (42.9%) |
| (N = 7) | | | | | | |
| ACA SSc | 3 (60.0%) | 2 (40.0%) | 3 (60.0%) | 2 (40.0%) | 2 (40.0%) | 2 (40.0%) |
| (N = 5) | | | | | | |
| Total Ctrl | 0 (0.0%) | 0 (0.0%) | 0 (0.0%) | 0 (0.0%) | 0 (0.0%) | 0 (0.0%) |
| (N = 18) | | | | | | |
| P values* | 1.4 × 10⁻⁴ | 3.9 × 10⁻⁴ | 3.9 × 10⁻⁴ | 3.9 × 10⁻⁴ | 3.9 × 10⁻⁴ | 3.9 × 10⁻⁴ |

*Comparisons are between all patients with SSc and all Controls (Ctrls).

TABLE 3
Results on validation of the 6 candidates by ELISA

| | FGF2 | | AIF1 | | EphB2 | | CLK1 | | THEX1 | | ANKS6 | |
|---|---|---|---|---|---|---|---|---|---|---|---|---|
| Positive/Total SSc tested (%) | 7/58 | (12%) | 36/88 | (41%) | 21/83 | (25%) | 21/62 | (34%) | 42/61 | (69%) | 31/56 | (55%) |
| Positive/Total tested | 2/18 | (11%) | 10/31 | (32%) | 4/28 | (14%) | 8/20 | (40%) | 12/18 | (67%) | 8/17 | (47%) |
| Ab^{neg}SSc (%) | | | | | | | | | | | | |
| Positive/Total tested ATA SSc (%) | 3/23 | (13%) | 21/36 | (58%) | 9/32 | (28%) | 9/25 | (36%) | 19/26 | (73%) | 17/22 | (77%) |
| Positive/Total tested ACA SSc (%) | 2/16 | (13%) | 5/20 | (25%) | 4/21 | (20%) | 5/21 | (24%) | 11/16 | (69%) | 6/16 | (38%) |
| Positive/Total tested Controls (%) | 8/110 | (7%) | 37/173 | (21%) | 24/172 | (14%) | 26/139 | (19%) | 29/123 | (24%) | 50/114 | (44%) |
| Positive/Total tested other AID (%) | 5/54 | (9%) | 23/91 | (25%) | 13/89 | (15%) | 16/74 | (21%) | 19/62 | (31%) | 31/57 | (54%) |
| Positive/Total tested healthy Ctrls | 3/56 | (5%) | 13/83 | (16%) | 11/83 | (13%) | 10/65 | (15%) | 10/61 | (16%) | 19/57 | (33%) |
| (%) | | | | | | | | | | | | |

| | | | | | | | |
|---|---|---|---|---|---|---|---|
| P values | [SSc vs all Ctrl] | ns* | 5.4 × 10⁻⁴ | 2.6 × 10⁻² | 1.9 × 10⁻² | 2.9 × 10⁻⁹ | ns |
| | [SSc vs other AID] | ns | 2.6 × 10⁻² | ns | ns | 2.2 × 10⁻⁵ | ns |
| | [SSc vs Healthy ctrls] | ns | 2.6 × 10⁻⁴ | 4.9 × 10⁻² | 1.5 × 10⁻² | 3.9 × 10⁻⁴ | 3.9 × 10⁻⁴ |

*ns: not significant

### EXAMPLE

### Material & Methods

Participants' Characteristics

Protein array analysis was made on plasma samples from 20 patients with SSc including 8 patients negative for ATA and ACA (Abneg), 6 positive for ATA and 6 positive for ACA recruited from 5 French hospitals. Patients with SSc were compared to 18 controls, including 8 healthy individuals with no history of autoimmune diseases (AID) recruited at the Centre d'Examen de Santé de l'Assurance Maladie (CESAM), Marseille, France and 10 patients with other AID including Rheumatoid Arthritis (RA), Systemic Erythematous Lupus (SLE) and Localized Scleroderma (LocSc) recruited in the Rheumatology Unit of St Marguerite Hospital in Marseille. The patients' autoantibody profile (ATA, ACA, Abneg) and patients' disease subtype (Lc-SSc, Dc-SSc) was obtained by reviewing medical records.

Each candidate was then tested on a larger cohort of minimum and maximum: 56 to 88 patients with SSc, 57 to 91 patients with other AID and 57 to 83 healthy controls. Distribution within each autoantibody group (Abneg, ATApos, ACApos) is detailed in results for each protein tested.

It is to note that Abneg patients are negative for ATA and ACA but could be positive for other autoantibodies (anti-RNA polymerase III, anti-U3RNP . . . ) but this was not reported in medical records.

Ethics Statements

All participants signed informed consent according to the Declaration of Helsinki (6). The study is registered at the INSERM under the Biomedical Research Protocol number RBM-04-10 or as a collection registered under the number DC-2008-327.

Detection of Autoantibodies by Protein Arrays

ProtoArray human protein microarrays V5.0 (Invitrogen, Carlsbad, Calif., USA) are spotted in duplicate on a nitrocellulose-coated glass slide with 9483 human proteins expressed using a baculovirus expression system, purified from insect cells (protein content list 5.0). Arrays were first blocked to avoid non-specific hybridization with Blocking Buffer (1% BSA, 1×PBS, 0.1% Tween® 20) at 4° C. for 1 hour (PartnerChip, Evry, France). Plasma samples, diluted 1:500 in Probe Buffer (1×PBS, 5 mM MgCl₂, 0.5 mM DTT, 5% glycerol, 0.05% Triton® X-100, 1% BSA) were added to arrays and incubated for 90 minutes at 4° C. in an incubation/hybridization chamber. Arrays were then washed 3 times for 8 minutes with 20 ml Probe Buffer, before adding a 1.0 μg/ml solution of anti-human IgG conjugated to Alexa Fluor® 647 (Invitrogen, Carlsbad, Calif., USA) for 90 minutes at 4° C. Arrays were washed again 3 times as described above and dried at room temperature. Arrays were scanned with a NimbleGen MS 200 scanner (Roche, Basel, Switzerland). Fluorescence data acquired with GenePix Pro Software and processed using Protoarray Prospector 5.2 (Invitrogen, Carlsbad, Calif., USA). Two control slides without plasma were used allowing the exclusion of 58 non-specific proteins from the 9483 spotted proteins (data not shown).

ProtoArray® Data Analysis

The ProtoArray® Prospector software includes a linear normalization algorithm that facilitates inter-assay data analysis and M-statistics algorithms for cross-group comparisons important for biomarker identification. These statistical tools allow comparing results between pairs of groups to identify probes which have consistently increased signals in the group of patients with SSc with respect to the control group (healthy individuals and patients with other AID).

Detection of Autoantibodies by ELISA

Plates were coated overnight at 4° C. with 0.2 μg of candidate protein per well diluted in PBS (except ANKS6 for which working conditions were defined at 0.1 μg/well). Plates were blocked with PBS 1% BSA overnight. After blocking solution removal, plasma samples diluted to 1:100 were added. After 2 hours of incubation at room temperature, plates were washed 3 times (1 minute) with PBS 0.1% Tween 20 and peroxidase-conjugated anti-human IgG (Sigma, France) was added for half an hour and then revealed with tetramethylbenzidine (TMB) liquid substrate system (Sigma, St Louis, Mo., USA). Optical density (OD) was read at 405 nm on a PowerWave XS microplate spectrophotometer (BIOTEK, France). For each individual, background OD was obtained by adding plasma on duplicated wells without tested protein. Positive plasmas were defined by an OD value superior or equal to twice the background OD (positive ΔOD=0 or more).

ELISA Data Analysis

To determine whether a candidate protein was significantly better recognized by plasmas from patients with SSc than healthy controls and/or patients with other AID, p values were calculated using the χ² test. For titration p values were evaluated using ANOVA test (Graphpad Prism 5).

Results

Plasma Samples from Patients with SSc Recognize More Proteins than Control Plasma Samples.

On ProtoArray® assays, plasma samples from patients with SSc (N=20) were the most reactive as they recognized in average, 4221 out of the 9483 human proteins (44.5%) compared to 1685 (17.8%) and 3080 (32.5%) proteins in respectively healthy controls (N=8) and patients with other AID (N=10) (Table 1). Differences in recognition (Mann Whitney test) were significant between patients with SSc and healthy controls (p=0.0009) and marginally significant between patients with SSc and patients with other AID (p=0.058).

ProtoArrays Identify Previously Described SSc Specific Autoantigens.

ATA and ACA are the most specific and the most often tested autoantibodies in SSc. As we had clinical records with ACA and ATA evaluation for the 20 patients tested (Supplementary Table Si), we compared them with ProtoArray results to validate the method. Twelve patients with SSc out of 20 (60%) and 5 controls out of 18 (28%) had anticentromere (CENP-B) antibodies by ProtoArrays, whereas based on clinical files, only 6 out of 20 patients with SSc (30%) were ACA positive. CENP-B was recognized by 5 of the 6 ACA positive as expected, but also by 5 of the 8 Ab negative and 2 of the 6 ATA positive patients (data not shown).

Among the 5 controls positive for CENP-B, two were healthy, one had Raynaud syndrome and has very recently received diagnosis of undifferentiated connective tissue disease and the three others were: 1 patient with RA, 1 with SLOC and 1 with SLE.

Topoisomerase I (Scl-70), a classical target in scleroderma, was recognized on ProtoArrays by 14 patients with SSc (3/8 Abneg, 6/6 ATA and 5/6 ACA) and 2 controls (1 patient with localized scleroderma and 1 healthy individual), whereas only 6 were positive for ATA from medical records.

Both topoisomerase and CENP-B were recognized with higher sensitivity in ProtoArrays than with classical laboratory clinical tests.

Identifying Antigens of Interest for Scleroderma by ProtoArray® Analysis.

Interpreting results from ProtoArray® assays requires setting the right cutoff for positive signal. The most stringent restriction would be choosing only proteins recognized by 100% of patients with SSc and none of the controls (data not shown) such ideal specific SSc candidate is not present in our assay. Moreover, under such stringent restrictions, neither topoisomerase I, nor centromere B would have been discovered by ProtoArrays.

Still, in order to validate highly specific and new candidates for SSc, we arbitrarily focused on proteins that were recognized by at least half of the patients with SSc and none of the controls. This reduced their number to 6 candidates (Table 2): Fibroblast Growth Factor 2 (FGF2), Allograft Inflammatory Factor 1 (AIF1) transcript variant 1, Ephrin Type B-receptor 2 (EphB2), Dual specificity protein kinase CLK1, Three prime Histone mRNA EXonuclease 1 (THEX1), Ankyrin repeat and Sterile alpha motif domain containing 6 (ANKS6).

Among those proteins, THEX1 seemed to be the most interesting as a new biomarker for patients without ACA and ATA as it was recognized by 62.5% of such patients (Table 2).

2 Candidates, AIF1 and THEX1, Confirmed by ELISA.

Candidate proteins were purchased from Invitrogen, except FGF2 which was purchased from Millipore. Proteins were plated on 96 well plates to be tested on a larger number of individuals by ELISA (Table 3).

FGF2, EphB2 CLK1 and ANKS6 were not specific for SSc as they were recognized by plasma samples from respectively 12%, 25%, 34% and 55% of patients with SSc and 9%, 15%, 21% and 54% of patients with other AID (Table 3).

Conversely, THEX1 and AIF1 were respectively recognized by 69% and 42% of plasma from patients with SSc and only 31% (p=0.00002) and 25% (p=0.0014), of plasma samples from other AID and 16% and 18% of plasma samples from healthy individuals (p=0.0004 and 0.0007 respectively). The initial ProtoArray's observation that THEX1 could be a good marker for patients without ACA or ATA was confirmed on a larger analysis, as this marker was recognized by 67% of Ab negative patients.

Although purified proteins plated for ELISA were warranted by the company to be the same as on microarrays, 4 of them specific for SSc by ProtoArrays did not remain specific for SSc by ELISA A possible explanation for differences in specificity between the same proteins in 2 different assays is differences in protein conformation. It is to note that the majority of human proteins spotted on the microarrays are expressed at high levels in insect cells which, similar to mammalian cells, proceed to protein folding and post-translational modifications such as phosphorylation and glycosylation [Augustin H G et al., 2003], in contrast to proteins expressed in E. coli. However among the 6 candidates for SSc, FGF2 and CLK1 were expressed in E. Coli. Moreover, FGF2 E. Coli-expressed purchased from Invitrogen did not work in our hands whereas FGF2 expressed in yeast (suitable for posttranslational modifications) purchased from Millipore did, although was not specific to SSc. CLK1 tested in ELISA was the exact same than the one on ProtoArray and did not give the conclusive results we had observed on ProtoArrays. If protein conformation plays a role in differences in results between the two assays, it would be in part solved with epitope mapping studies.

Therefore, the 4 proteins found by ProtoArrays but unvalidated by ELISA should still be considered as biomarkers for the diagnosis of scleroderma and particularly for systemic sclerosis.

AIF1 and THEX1 ELISA Titers.

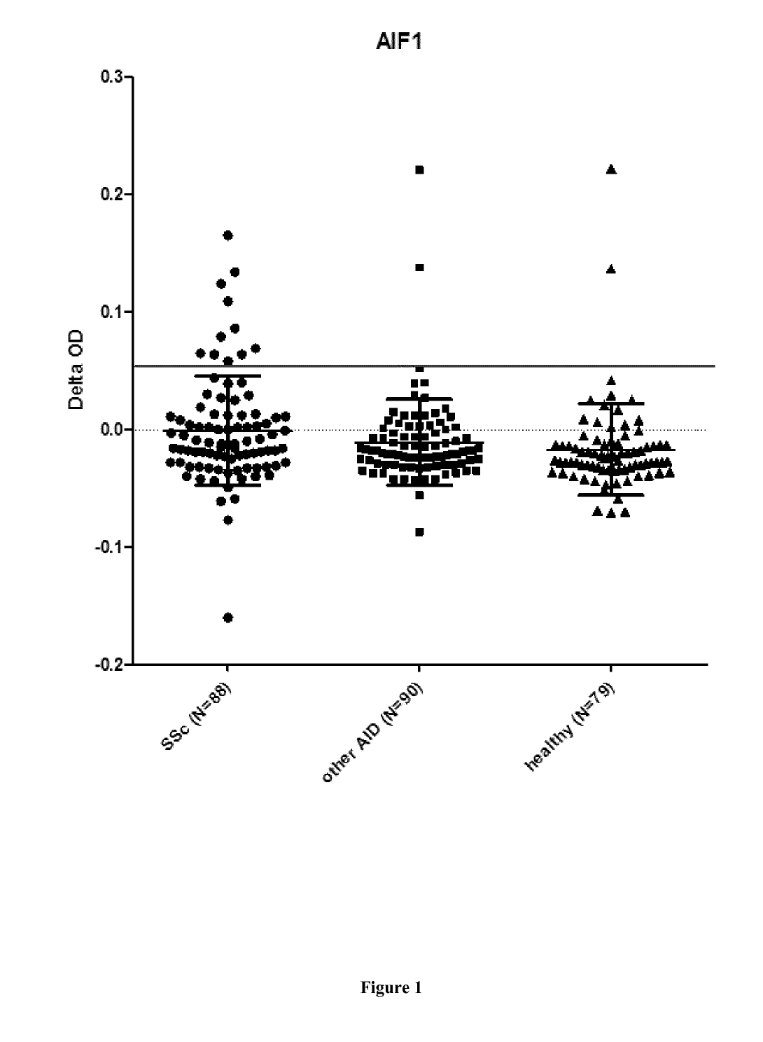

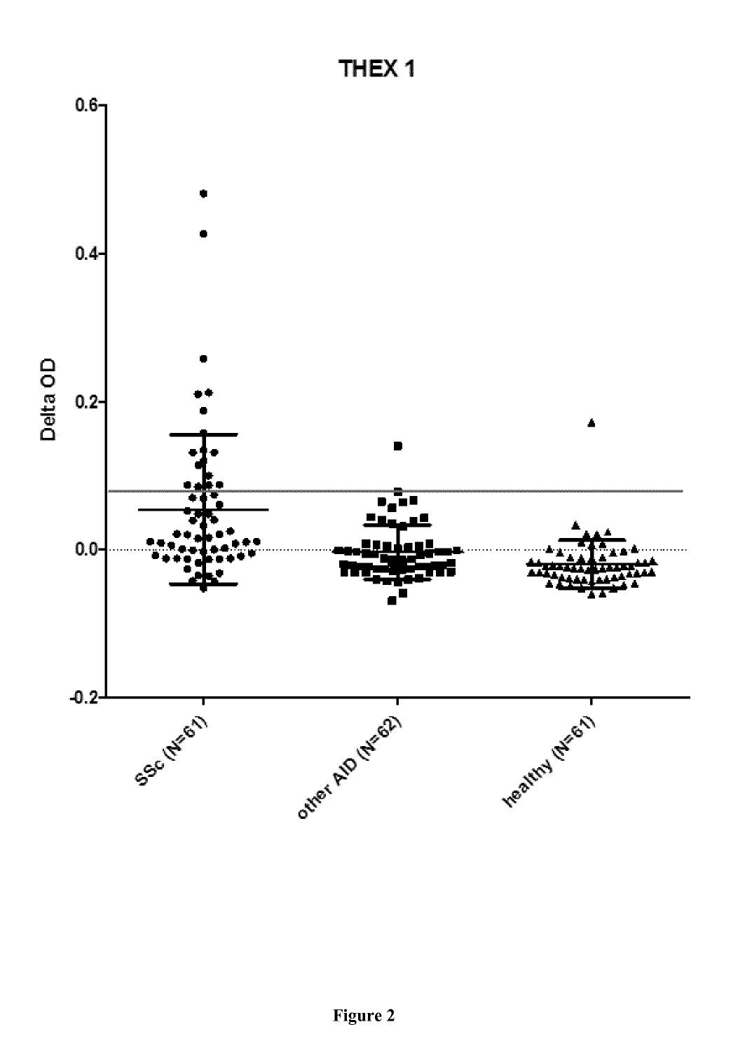

We further analyzed differences in titers for AIF1 and THEX1 between SSc and other groups for diagnostic tool development purpose. For AIF1, ELISA titers were marginally higher in SSc compared to other control groups (p=0.04, FIG. 1). However setting a threshold at ΔOD=0.055 would allow detection of 11/88 (12.5%) patients with SSc and 4/169 (2.4%) of pooled controls (healthy controls and patients with other AID) (p=0.001). For THEX1, titers were statistically higher in SSc compared to control groups (<0,001) (FIG. 2). A threshold of ΔOD at 0.08 allows detection of 17/61 (11.5%) patients with SSc and 2/123 (1.6%) of pooled control groups (p<10-7).

### REFERENCES

Throughout this application, various references describe the state of the art to which this invention pertains.
Alkassab F, Gourh P, Tan F K, McNearney T, Fischbach M, Ahn C, Arnett F C, Mayes M D. An allograft inflammatory factor 1 (AIF1) single nucleotide polymorphism (SNP) is associated with anticentromere antibody positive systemic sclerosis. Rheumatology (Oxford). 2007 August; 46(8):1248-51. Epub 2007 May 23.
Allanore Y, Cabane J, Mouthon L (2007) Sclérodermies. In: Med-line, editor. Classification et evaluation des sclerodermies. Paris.
Arnett F C, Gourh P, Shete S, Ahn C W, Honey R E, et al. (2010) Major histocompatibility complex (MHC) class II alleles, haplotypes and epitopes which confer susceptibility or protection in systemic sclerosis: analyses in 1300 Caucasian, African-American and Hispanic cases and 1000 controls. Ann Rheum Dis 69: 822-827.
Augustin H G, Reiss Y. EphB receptors and ephrinB ligands: regulators of vascular assembly and homeostasis. Cell Tissue Res. 2003; 314(1):25-31.
Hachulla Eric & David Launay. Diagnosis and Classification of Systemic Sclerosis. Clinic Rev Allerg Immunol (2011) 40:78-83.
Ho K T, Reveille J D (2003) The clinical relevance of autoantibodies in scleroderma. Arthritis Res Ther 5: 80-93.
Oddis C V, Okano Y, Rudert W A, Trucco M, Duquesnoy R J, Medsger Jr T A. Serum autoantibody to the nucleolar antigen PM-Scl. Clinical and immunogenetic associations. Arthritis Rheum 1992; 35 (10): 1211-7.
Okano Y, Steen V D, Medsger Jr T A. Autoantibody reactive with RNA polymerase III in systemic sclerosis. Ann Intern Med 1993; 119(10): 1005-13.
Okano Y, Steen V D, Medsger Jr T A. Autoantibody to U3 nucleolar ribonucleoprotein (fibrillarin) in patients with systemic sclerosis. Arthritis Rheum 1992; 35(1):95-100.
Okano Y, Medsger Jr T A. Autoantibody to Th ribonucleoprotein (nucleolar 7-2 RNA protein particle) in patients with systemic sclerosis. Arthritis Rheum 1990; 33(12):1822-8.
Reveille J D (2006) The genetic basis of autoantibody production. Autoimmun Rev 5: 389-398.
Steen V D, Powell D L, Medsger Jr T A. Clinical correlations and prognosis based on serum autoantibodies in patients with systemic sclerosis. Arthritis Rheum 1988; 31(2):196-203.
Walker U A, Tyndall A, Czirjak L, Denton C P, Farge Bancel D, et al. (2007) Clinical risk assessment of organ manifestations in systemic sclerosis—a report from the EULAR Scleroderma Trials And Research (EUSTAR) group data base. Ann Rheum Dis.

1. An in vitro method for diagnosing scleroderma in a subject, said method comprising the step of detecting in a biological sample obtained from the subject one or more autoantibodies recognizing one or more protein biomarkers selected from the group of proteins consisting of THEX1, AIF1, FGF2, EphB2, CLK1 and ANKS6.

2. An in vitro method according to the claim 1 wherein the scleroderma is a systemic sclerosis.

3. The method according to claim 2 wherein said method comprises the steps of:
contacting a biological sample obtained from the subject with one, two, three, four, five or six protein biomarkers selected from the group consisting of THEX1, AIF1, FGF2, EphB2, CLK1 and ANKS6; and
detecting any biomarker-antibody complex formed, wherein the detection of a biomarker-antibody complex is indicative of systemic sclerosis in the subject.

4. The method according to claim 3, wherein said biological sample obtained from the subject is contacted with the two protein biomarkers: THEX1 and AIF1.

5. The method according to claim 3, wherein said biological sample obtained from the subject is contacted with said 6 six protein biomarkers.

6. A kit for the in vitro diagnosis of scleroderma in a subject, said kit comprising one, two, three, four, five or six protein biomarkers selected from the group consisting of THEX1, AIF1, FGF2, EphB2, CLK1 and ANKS6; and
at least one reagent for detecting a biomarker-antibody complex formed between the protein biomarker and an autoantibody present in a biological sample obtained from the subject.

7. The kit according to claim 6, wherein said kit comprises said six protein biomarkers.

8. The kit according to claim 6 wherein said kit comprises the two protein biomarkers: THEX1 and AIF1.

9. An array for diagnosing of scleroderma in a subject, said array comprising, attached to a surface., one, two, three, four, five or six protein biomarkers selected from the group consisting of THEX1, AIF1, FGF2, EphB2, CLK1 and ANKS6.

10. The array according to claim 9, said array comprising, attached to the surface said six protein biomarkers.

11. The array according to claim 9, said array comprising, attached to the surface, the 2 protein biomarkers: THEX1 and AIF1.


SEQUENCE LISTING (6 sequences)

SEQ ID NO: 1 - protein, 349 aa, Homo sapiens
MEDPQSKEPAGEAVALALLESPRPEGGEEPPRPSPEETQQCKFDGQETKGSKFITSSASDFSDPVYKEIAITNGCINRMSKEELRAKLSEFKLETRGVKDVLKKRLKNYYKKQKLMLKESNFADSYYDYICIIDFEATCEEGNPPEFVHEIIEFPVVLLNTHTLEIEDTFQQYVRPEINTQLSDFCISLTGITQDQVDRADTFPQVLKKVIDWMKLKELGTKYKYSLLTDGSWDMSKFLNIQCQLSRLKYPPFAKKWINIRKSYGNFYKVPRSQTKLTIMLEKLGMDYDGRPHCGLDDSKNIARIAVRMLQDGCELRINEKMHAGQLMSVSSSLPIEGTPPPQMPHFRK

SEQ ID NO: 2 - protein, 93 aa, Homo sapiens
MEFDLNGNGDIDIMSLKRMLEKLGVPKTHLELKKLIGEVSSGSGETFSYPDFLRMMLGKRSAILKMILMYEEKAREKEKPTGPPAKKAISELP

SEQ ID NO: 3 - protein, 155 aa, Homo sapiens [sequence omitted]

SEQ ID NO: 4 - protein, 286 aa, Homo sapiens
HNQTSLYKKAGSEIDISCVKIEQVIGAGEFGEVCSGHLKLPGKREIFVAIKTLKSGYTEKQRRDFLSEASIMGQFDHPNVIHLEGVVTKSTPVMIITEFMENGSLDSFLRQNDGQFTVIQLVGMLRGIAAGMKYLADMNYVHRDLAARNILVNSNLVCKVSDFGLSRFLEDDTSDPTYTSALGGKIPIRWTAPEAIQYRKFTSASDVWSYGIVMWEVMSYGERPYWDMTNQDVINAIEQDYRLPPPMDCPSALHQLMLDCWQKDRNHRPKFGQIVNTLDKMIRNPN

SEQ ID NO: 5 - protein, 600 aa, Homo sapiens
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRPWSNQTSLYKKAGFEGDRTMHGKSHRRKRTRSVEDDEEGHLICQSGDVLSARYEIVDTLGEGAFGKVVECIDHKAGGRHVAVKIVKNVDRYCEAARSEIQVLEHLNTTDPNSTFRCVQMLEWFEHHGHICIVFELLGLSTYDFIKENGFLPFRLDHIRKMAYQICKSVNFLHSNKLTHTDLKPENILFVQSDYTEAYNPKIKRDERTLINPDIKVVDFGSATYDDEHHSTLVSTRHYRAPEVILALGWSQPCDVWSIGCILIEYYLGFTVFPTHDSKEHLAMMERILGPLPKHMIQKTRKRKYFHHDRLDWDEHSSAGRYVSRRCKPLKEFMLSQDVEHERLFDLIQKMLEYDPAKRITLREALKHPFFDLLKKSI

SEQ ID NO: 6 - protein, 241 aa, Homo sapiens [sequence omitted]